(19) United States
(12) Patent Application Publication
(10) Pub. No.: US 2010/0016817 A1
(43) Pub. Date: Jan. 21, 2010

(54) Bedside suction cup

(76) Inventor: Arlette M. Cadoche Jacobson, Silver Spring, MD (US)

Correspondence Address: Arlette M. Cadoche Jacobson, 11606 Fulham St., Silver Spring, MD, 20902, US

(21) Appl. No.: 12/453,748

(22) Filed: May 21, 2009

Related U.S. Application Data
(60) Provisional application No. 61/129,262, filed on Jun. 16, 2008.

(51) Int. Cl. A61M 1/00

(52) U.S. Cl. 604/319; 604/323

(57) ABSTRACT

A suction cup apparatus includes a container body and a valve which covers the container body. The valve has an X-shaped slit therein for resiliently securing a suction catheter. A support member is provided for holding the container body. A flexible arm is also provided, having a first end and a second end, the first end being connected to the support member. A clamp disposed on the second end of the flexible arm for removably securing the flexible arm to a support. In use, the suction cup preferably contains a sterile solution made of water and antibacterial solution with or without a flavoring agent The volume of the suction cup is preferably in the range of 12 oz to 20 oz. But can vary according to use.

### CROSS REFERENCE TO RELATED APPLICATIONS

This application claims priority to Provisional No. 61/129,262 filed on Jun. 16, 2008, inventor Arlette Cadoche-Jacobson, entitled “Bedside Suction Cup”

### STATEMENT REGARDING FEDERALLY SPONSORED RESEARCH OR DEVELOPMENT NOT APPLICABLE

### FIELD OF THE INVENTION

The present invention relates generally to an apparatus for providing and supporting a bedside suction cup, and a novel suction cup arrangement. More particularly, this invention is directed to an apparatus or device for providing and supporting a bedside suction cup usefull for medical and treatment procedures and treatment of the body, mouth and trachea area for patient in hospital, nursing home or home care situations.

### BACKGROUND OF THE INVENTION

As noted in U.S. Pat. No. 4,925,447 to Rosemblat, an aspirator and a container are shown for use with a body of a patient, for removing muccus or other body fluids. FIG. 8 thereof shows a yankauer suction tip which is replaceable.

In U.S. Pat. No. 5,823,657 to Price et al shows a lamp clamp supporting a flash light with a flexible stem such as a snake like flashlight.

U.S. Pat. No. 5,033,655 to Brown teaches a dispensing package for fluid product and shows a flexible valve having an x-shaped slit in FIG. 37 thereof. There is a need for an apparatus or a device for providing and supporting a bedside suction cup usefull for suction of the body, mouth and trachea areas for patients in hospital, nursing home or home, care situations.

### SUMMARY OF THE INVENTION

From the foregoing, it is seen that it is a problem in the art to provide a device meeting the above requirements.

According to the present invention, a device is provided which meets the aforementioned requirements and needs in the prior art. Specifically, the device according to the present invention providing and supporting a bedside suction cup usefull for medical procedures and treatments of the body, mouth, and trachea areas for patients in hospital or in home care situations.

The device according to the present invention includes a novel suction cup having a flexible valve, and a valve cover. The suction cup can thereby dispense sterile or clean liquids from the suction cup, and can be used in a clean maner multiples times. It is recommended to change the apparatus daily for clean use. For sterile use, open a new suction cup each time. In use, an IV stand is provided near a patient, and a clamp is used to secure a suction cup in a convenient location such as bed board or wall for providing liquids used in medical procedures and treatments. The suction cup is a sterile/clean container having a recloseable lid and a flexible valve member for holding a patient suction catheter therein.

Other objects and advantages of the present invention will be more readilly apparent from the following detailed description when read in conjunction with the accompanying drawings.

### BRIEF DESCRIPTION OF THE DRAWINGS

FIG. 1 is a schematic side view of a suction cup apparatus and arrangement, according to the present invention.

FIG. 2 is a top elevational view of the suction cup of the arrangement of FIG. 1.

FIG. 3 is an enlarged top elevational view of the flexible valve protion of the FIG. 2.

FIG. 4 is a side elevational view of the flexible valve protion of FIG. 3

FIG. 5 is a side sectional view taken along line 5-5 of FIG. 3.

FIG. 6 is a top elevational view of a flexible valve structure usable in the suction cup of FIG. 1.

FIG. 7 is a sectional view taken through the center of the FIG. 6, showing concave portions of the central area thereof.

FIG. 8 is a schematic side view of the valve and hinged cover portion of the FIG. 1

FIG. 9 is a side elevational view of a yankauer tip having a sleeve.

### DETAILED DESCRIPTION OF THE INVENTION

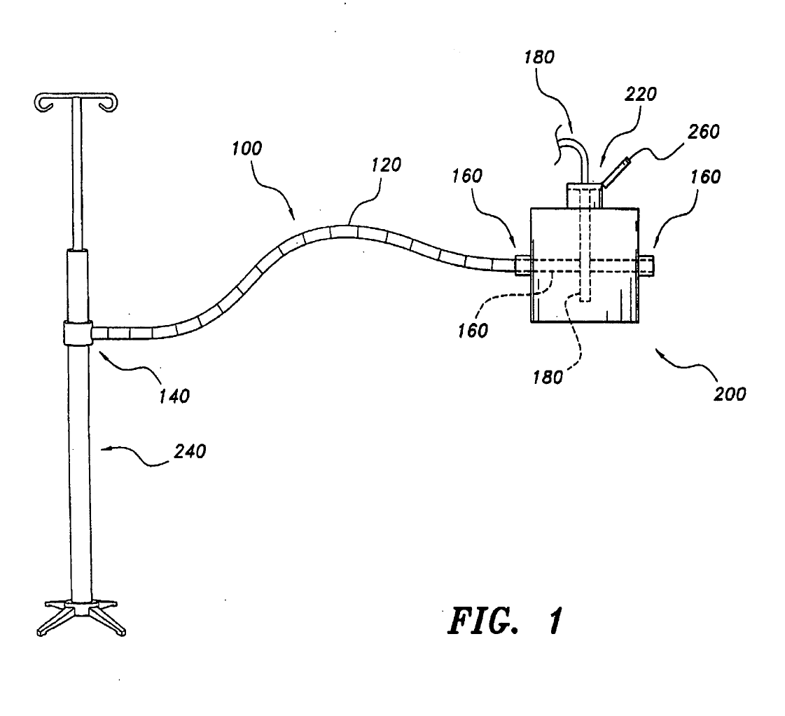

FIG. 1 is a schematic side view of a suction cup apparatus 100. The apparatus includes a flexible repositionable arm 120 which can be attached to an IV pole 240 (or a bed board or wall apparatus) by any conventional clamp 140 such as a spring-loaded clip-type of clamp. The flexibly repositionable arm 120 is securely affixed to a securing ring 160 which supports a suction cup 200. A valve 220 is at the top of the suction cup 200 and allowes passage there through of a patient catheter 180. A hinged closure member 260 is attached to the top of the suction cup 200 for selectively covering and uncovering the valve 220.

In use, the suction cup 200 preferably contains a sterile solution made of water and antibacterial solution with or without a flavoring agent. The volume of the suction cup 200 is preferably in the range of 12 oz. to 20 oz. but can vary according to use.

The suction cup 200 may be of various forms including but not limited to square, oval, round, triangular, and so on, and may be redesigned to fit the needs of the patient, or for commercial purposes. The material forming the body of the suction cup 200 is preferably of disposable plastic material with an opening on the top for the valve 220 and hinged closure member 260. The valve 220 is preferably made of durable plastic which is sufficiently strong to hold a suction catheter 180 in place.

The suction cup 200 may be attached to a bed, wall, or IV pole 240, via the flexibly repositionable arm 120. The flexibly repositionable arm 120 is preferably formed of interlocked rings, similar to that used for the flexible arm of the well known prior art device called a snakelight flashlight.

The suction cup 200 is preferably disposable and will be changed daily, and may not be sterile if suctioning mouth secretions. The clamp 140 is preferably large enough to allow positioning at a bed rail or ventilator or wall mount as well at the IV pole 240, or at foot/head of the bed according to the patient's comfort and mobility.

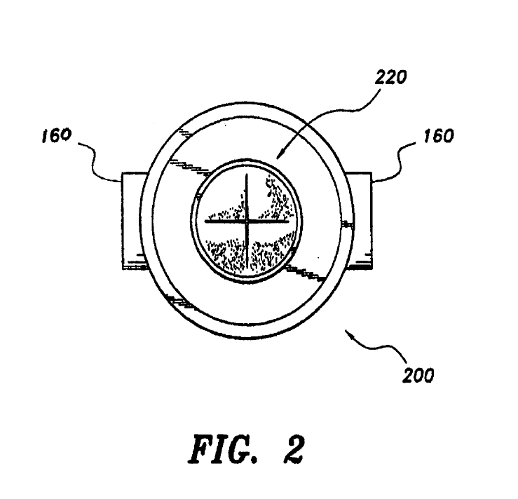

FIG. 2 is a top elevational view of the suction cup 200 for the arrangement of FIG. 1. The securing ring 160 is seen, as well as the flexible valve 220.

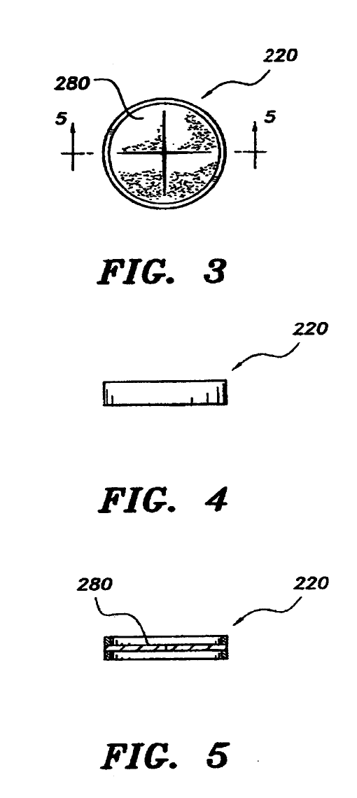

FIG. 3 is an enlarged top elevational view of the flexible valve 220 of FIG. 2. the valve 220 includes a central flexible valve portion 280.

FIG. 4 is a side elevational view of the flexible valve 220 of FIG. 3.

FIG. 5 is a side sectional view taken along the lines 5-5 of FIG. 3. Here, the central valve portion 280 is a schematically shown.

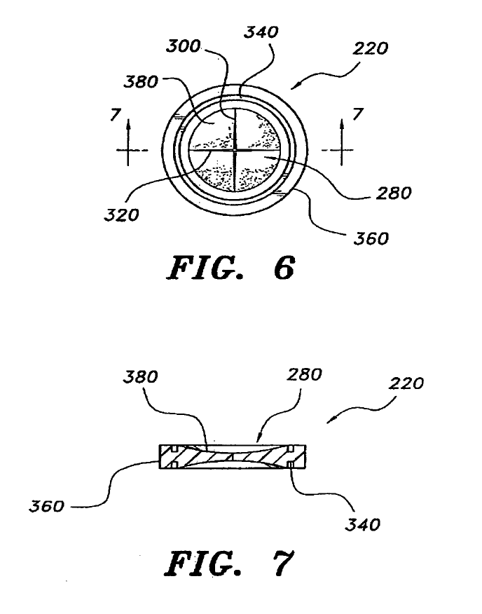

FIG. 6 is a top elevational view of a flexible valve structure usable in the suction cup 200 of FIG. 1. In particular, the valve 220 has a pair of slits 300, 320 arranged in an x-shape 380, so as to flexibly and resiliently secure a suction catheter or a patient tube therein. The central valve portion 280 has an outermost annular valve portion 360 and an inner annular valve support portion 340, which in turn surrounds the x-shape 380 of the central valve portion 280.

FIG. 7 is a sectional view taken through the center of FIG. 6, showing concave protions 380 of the central area thereof.

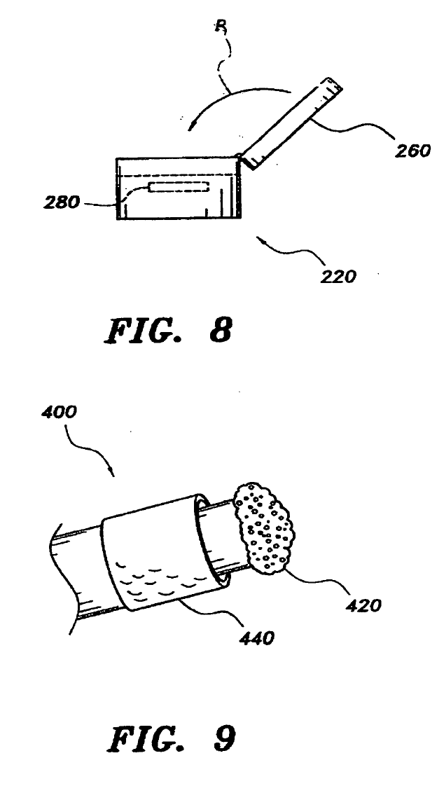

FIG. 8 is a schematic side view of the valve and hinged cover portion 260 of FIG. 1. The hinged closure member 260 closes in the direction indicated by the arrow labelled “r” in this view.

FIG. 9 is a side elevational view of a suction catheter tip 400 having an end 420 having passageways therein for passage of liquids, and a sleeve 440 for gripping and cleaning. The sleeve 440 can be made of material such as that used in dental accessories for cleaning of the tongue or gums.

The invention being thus described, it will be evident that the same may be varied in many ways by routineer in the applicable arts. Such variations are not to be regarded as a departure from the spirit and scope of the invention and all such modifications are intended to be included within the scope of the claims.

What is claimed is:

1. A suction cup apparatus, comprising:
a container body and a valve which covers the container body; the valve having an x-shaped slit therein for resiliently securing a catheter or a patient tube therein; a support member for holding the container body; a flexible arm having a first end and a second end, the first end being connected to the support member; and a clamp disposed on the second end of the flexible arm for removably securing the flexible arm to a support.

2. A suction cup apparatus as in claim 1, wherein the support member is a ring-shaped member.

3. A suction cup apparatus as claimed in claim 2, wherein the valve has an inner annular valve support portion and a outermost annular valve portion.

4. A suction cup apparatus as claimed in claim 1, wherein said flexible arm is composed of metal.

5. A suction cup apparatus as claimed in claim 1, wherein said container body is composed of plastic.

6. A suction cup apparatus as claimed in claim 1, wherein said container body is sufficiently large to hold 20 ounce of fluid.

7. A suction cup apparatus as claimed in claim 1, wherein said container body has a hinged cover which covers the valve.
